(19) United States
(12) Patent Application Publication
(10) Pub. No.: US 2013/0165514 A1
(43) Pub. Date: Jun. 27, 2013

(54) ORALLY ADMINISTERED COMPOSITION AND DIETARY COMPOSITION HAVING SERUM LIPID IMPROVING EFFECT

(71) Applicants: Kenji Fukunaga, Kyoto (JP); Yoshio Shimizu, Okayama (JP); Mikiya Takamoto, Okayama (JP); Teruyuki Kanada, Okayama (JP)

(72) Inventors: Kenji Fukunaga, Kyoto (JP); Yoshio Shimizu, Okayama (JP); Mikiya Takamoto, Okayama (JP); Teruyuki Kanada, Okayama (JP)

(73) Assignee: BIZEN CHEMICAL CO., LTD., Okayama (JP)

(21) Appl. No.: 13/816,627

(22) PCT Filed: Feb. 18, 2011
(86) PCT No.: PCT/JP2011/000930

§ 371 Date: Mar. 7, 2013

(30) Foreign Application Priority Data
Aug. 31, 2010 (JP) 2010-195157

(51) Int. Cl. A61K 31/232

(52) U.S. Cl. CPC A61K 31/232
USPC 514/549

(57) ABSTRACT

The present invention provides an orally administered composition and a dietary composition, which have a serum lipid improving effect. The orally administered composition and dietary composition, having a serum lipid improving effect, according to the present invention camp rise 2.0% by weight or less of a DHA ethyl ester composition comprising 75% by weight or more of DHA ethyl ester. It is preferable to comprise 1.2% by weight or less of the DHA ethyl ester composition. It is preferable not to substantially comprise a DHA ethyl ester composition comprising 30% by weight or less of DHA ethyl ester.

### TECHNICAL FIELD

The present invention relates to an orally administered composition and a dietary composition, having a serum lipid improving effect. More particularly, the present invention relates to an orally administered composition and a dietary composition, comprising a small amount of high-purity docosahexaenoic acid ethyl ester, and used as a pharmaceutical or supplement.

### BACKGROUND ART

N-3 polyunsaturated fatty acids, such as docosahexaenoic acid (DHA) and eicosapentaenoic acid (EPA), characteristically comprised in marine products, have various types of bioactivity, such as an action for improvement in lipid metabolism, and an anti-atherogenic action. Thus, the n-3 polyunsaturated fatty acids are used as a pharmaceutical or supplement.

For example, Japanese Laid-Open Publication No. 2000-95683 (Patent Document 1) describes a skin external preparation comprising a docosahexaenoic acid ester derivative. It describes that the purity of the ester derivative of EPA or DHA is preferably high so as the dose can be small (paragraph 0006).

However, Patent Document 1 relates to a skin external preparation, and not to an orally administered composition or a dietary composition. Furthermore, Patent Document 1 does not disclose any serum lipid improving effect at all.

Japanese Laid-Open Publication No. 2000-44470 (Patent Document 2) describes a hyperlipidemia drug comprising a docosahexaenoic acid ester derivative. It describes that the purity of the docosahexaenoic acid ester derivative is preferably high so as the dose can be small (paragraph 0008),

However, Patent Document 2 does not concern the absorption of ester derivatives of eicosapentaenoic acid or docosahexaenoic acid of low purity; and it does not disclose a serum lipid improving effect.

### PRIOR ART DOCUMENTS

### Patent Documents

Patent Document 1; Japanese Laid-Open Publication No. 2000-95683

Patent Document 2: Japanese Laid-Open Publication No. 2000-44470

### SUMMARY OF INVENTION

### Problem To Be Solved By the Invention

The present invention is provided for solving the above-mentioned defects, and the purpose of the present invention is to provide an orally administered composition and a dietary composition, having a serum lipid improving effect.

### Means For Solving the Problem

The dietary composition according to the present invention has the following features.
(Item 1) A dietary composition having a serum lipid improving effect, the dietary composition comprising 2.0% by weight or less of a DHA ethyl ester composition comprising 75% by weight or more of DHA ethyl ester.
(Item 2) The dietary composition having a serum lipid improving effect according to Item 1, wherein the dietary composition comprises 1.2% by weight or less of the DHA ethyl ester composition.
(Item 3) The dietary composition having a serum lipid improving effect according to Item 1, wherein the dietary composition comprises 1.0% by weight or less of the DHA ethyl ester composition.
(Item 4) The dietary composition having a serum lipid improving effect according to Item 1, wherein the dietary composition does not substantially comprise a DHA ethyl ester composition comprising 30% by weight or less of the DHA ethyl ester.

The orally administered composition according to the present invention has the following features.
(Item 5) An orally administered composition having a serum lipid improving effect, the orally administered composition comprising 2.0% by weight or less of a DHA ethyl ester composition comprising 75% by weight or more of DH ethyl ester.
(Item 6) The orally administered composition having a serum lipid improving effect according to Item 5, wherein the orally administered composition comprises 1.2% by weight or less of the DHA ethyl ester composition.
(Item 7) The orally administered composition having a serum lipid improving effect according to Item 5, wherein the orally administered composition comprises 1.0% by weight or less of the DHA ethyl ester composition.
(Item 8) The orally administered composition having a serum lipid improving effect according to Item 5, wherein the orally administered composition does not substantially comprise a DHA ethyl ester composition comprising 30% by weight or less of the DHA ethyl ester.
(Item 9) The orally administered composition according to any of Items 5 to 8, which is a pharmaceutical composition.

The orally administered composition according to the Present.. invention described above is preferably a pharmaceutical composition.

### Effect of the Invention

When the purity of DHA is low, fatty acids other than DHA will undergo enzymatic degradation, and thereby DHA will be less likely to influence the action of the enzyme. However, the dietary composition or orally administered composition according to the present invention comprises 2.0% by weight or less of a DHA ethyl ester composition that comprises 75% by weight or more of DHA ethyl ester. Thus, high-purity DNA has more opportunities to be influenced by the action of enzyme, which increases the absorption rate of DHA. The dietary composition or orally administered composition according to the present invention has a serum lipid improving effect, an anti-inflammatory effect and a serum triglyceride reducing effect.

### BRIEF DESCRIPTION OF THE DRAWINGS

FIG. 1 is a graph, showing serum lipid contents.

FIG. 2 is a graph, showing serum HDL-C and LDL-C contents.

FIG. 3 is a graph, showing hepatic lipid contents.

FIG. 4 is a graph, showing respective fatty acid contents in total hepatic lipid.

FIG. 5 is a graph, showing respective fatty acid contents in total brain lipid.

FIG. 6 is a graph, showing DHA-EE (docosahexaenoic acid ethyl ester) excreted with feces

### EMBODIMENTS FOR CARRYING OUT THE INVENTION

Hereinafter, embodiments of the present invention will be described.

The orally administered composition and dietary composition according to the present invention comprises 2.0% by weight or less of a DHA ethyl ester composition comprising high-purity (i.e., comprising 75% by weight or more of) DNA ethyl ester. The orally administered composition according to the present invention is preferably a pharmaceutical composition.

In the DHA ethyl ester composition, the content of the DHA ethyl ester is preferably 80% by weight or more, and more preferably 90% by weight or more.

In addition, the orally administered composition and dietary composition according to the present invention preferably comprise 1.2% by weight or less of the DHA ethyl ester composition comprising high-purity DHA ethyl ester.

A dietary composition comprising a large amount of a DHA ethyl ester composition comprising low purity (i.e., comprising 30% by weight or less) of DHA ethyl ester has a low serum lipid improving effect. The reason for this is assumed that a DHA ethyl ester composition comprising a low purity DHA ethyl ester has a large amount of fatty acid ethyl ester, other than DHA, compared to a DHA ethyl ester composition comprising high-purity DHA ethyl ester, and that is why lipase is not able to react to the DHA ethyl ester (hereinafter, referred to as DHA-EE). Accordingly, in consideration of the absorption efficiency on the premise of the expression of functionality, it is more desirable to ingest a smaller amount of high-purity DHA-EE than to ingest a large amount of low purity DHA-EE.

With regard to the orally administered composition and dietary composition according to the present invention, the effective ingredient, DHA-EE, will be effectively absorbed when the composition comprises a small amount of high-purity DHA-EE, thus avoiding problems with side effects due to other ingredients comprised, and thus allowing the form of the formulation (e.g., soft capsule) per dosage to be small, which makes it easy to administer.

The orally administered composition and dietary Composition according to the present invention are formulated in the form of tablets, capsules, granules, powders or a liquid formulation.

For an orally administered carrier, an ordinarily used agent can be used such as emulsifier, excipient, binder, lubricant, colorant or disintegrator. For example, the excipient includes lactose, sucrose, starch, talc, magnesium stearate, crystalline cellulose, methyl cellulose, carboxy methyl cellulose, glycerin, sodium alginate, gum arabic and the like. The binder includes polyvinyl alcohol, polyvinyl ether, ethyl cellulose, gum arabic, shellac, saccharose and the like. In addition, publicly known colorants and disintegrators can be used. The tablets may be coated using any publicly known method. Furthermore, the liquid formulation may be an aqueous or oily suspension, emulsifier, solution, syrup, elixir and the like. The liquid formulation is formulated using an ordinarily used method.

The particularly preferable administration form of the orally administered composition and dietary composition according to the present invention is capsules and granules.

The orally administered composition and dietary composition according to the present invention is administered, either solely or together with a publicly known agent, to mammals such as humans, cows, horses, dogs, cats, mice, rats and the like. The human dose of the dietary composition according to the present invention is, without any particular limitation, preferably 0.1 to 10 g/day, and more preferably 0.5 to 5 g/day as DHA.

Hereinafter, the present invention will be described in more detail with reference to Examples or the like; however, the present invention will not be limited to those Examples.

### EXAMPLES

### Example 1

With regard to the influence by high-purity and low-purity DHA--EE with different DHA contents on lipid metabolism, rats were used as an experimental animal to examine influence of PEA content on serum, hepatic lipid contents, the liver and the brain.

### A. Experimental Method

5 week-old, Wistar male rats were fed with AIN93G with soybean oil (7%) as its lipid source (control), feed in which the soybean oil of AIN93G was substituted with 1% high-purity DHA-EE (75% DHA content) (DHA-75EE group), and feed with a substitution of 3% low-purity DHA-EE (25% DHA content) (DHA-25EE group), for 28 days.
The DHA content was adjusted to be equal in the DHA-75EE feed and DHA-25EE feed.
After the completion of the feeding period, the serums, livers, brains and white adipose tissues (WAT) were collected. The lipid components of the serums and livers as well as the amount of respective fatty acids in the total lipid of the livers and brains were measured. In addition, feces were collected for the last seven days of the feeding period to measure the amount of the DHA-EE excreted with the feces.

The compositions of the feed (adjusted with AIN93G as original) are as follows:

| Group | Lipid Source (content rate) |
|---|---|
| Soybean Oil (SO) | 7.0% Soybean Oil |
| Low-purity DHA-EE | 4.2% Soybean oil + 2.8% |
| | low-purity DHA-EE (DHA25EE) |
| High-purity DHA-EE | 6.0% Soybean oil + 1.0% |
| | High-purity DHA-EE (DHA75EE) |

The fatty acid composition (% by weight) in the DHA-75EE (high-purity DHA-EE) and the fatty acid composition (% by weight) in the DHA-25EE (low-purity DHA-EE) are shown on Table 1.

TABLE 1
Fatty Acid Composition in Respective DHA-EE (% by weight)

| | Low Purity DHA-EE | High Purity DHA-EE |
|---|---|---|
| 14:0 | 4.5 | N.D. |
| 16:0 | 25.3 | N.D. |
| 16:1 n-7 | 5.1 | N.D. |
| 18:0 | 6.1 | 0.3 |
| 18:1 n-9 | 12.9 | 1.1 |
| 18:1 n-7 | 2.3 | N.D. |
| 18:2 n-6 | 1.2 | N.D. |
| 18:3 n-3 | 0.6 | N.D. |
| 20:4 n-6 | 1.0 | 2.0 |
| 20:5 n-3 | 3.0 | 3.0 |
| 22:5 n-6 | 2.2 | 5.8 |
| 22:5 n-3 | 1.2 | 3.0 |
| 22:6 n-3 DHA | 26.7 | 75.4 |
| Others | 7.9 | 9.4 |

As shown in Table 1, the amounts of DHA administered were adjusted to be the same in the experiment. This was because the amounts of DHA would not be the same as the high-purity DHA ethyl ester product unless a large amount (about three times) of the low concentration DHA ethyl ester product would be administered.

The fatty acid compositions (% by weight) in the DHA-7SEE feed and DHA-25EE feed are as shown in Table 2.

TABLE 2
Fatty Acid Composition in Respective Feed (% by weight)

| | SO | DHA25EE | DHA75EE |
|---|---|---|---|
| 14:0 | N.D. | 1.5 | N.D. |
| 16:0 | 10.6 | 15.4 | 9.1 |
| 16:1 n-7 | N.D. | 2.0 | N.D. |
| 18:0 | 3.8 | 4.7 | 3.3 |
| 18:1 n-9 | 21.8 | 18.2 | 18.8 |
| 18:1 n-7 | 1.4 | 1.7 | 1.2 |
| 18:2 n-6 | 53.7 | 32.5 | 46.0 |
| 18:3 n-3 | 5.8 | 3.7 | 5.0 |
| 20:4 n-6 | N.D. | 0.4 | 0.3 |
| 20:5 n-3 | N.D. | 2.3 | 0.8 |
| 22:5 n-3 | N.D. | 0.9 | 0.8 |
| 22:5 n-6 | N.D. | 0.5 | 0.4 |
| 22:6 n-3 DHA | N.D. | 10.8 | 10.8 |
| Others | 2.9 | 5.3 | 3.4 |

### B. Analysis Method

(1) For the blood, the serum lipid component was measured using Olympus AU5431.
(2) For the liver, the following method was used:
Total Lipid: Bligh & Dyer method
Neutral Lipid: Enzymatic method
Cholesterol: 010 using internal standard
Phospholipid: molybdenum-ammonium method
(3) Growth parameters were set as fellows:
Final Weight (g)
Weight Gain (g/day)
Energy Intake (kcal/day)
Feed Efficiency (g/kcal)
Liver Weight (g/100 gBW)
Fatty Tissue Weight (g/100 gBW)

### C. Result

(1) No particular difference was observed in the growth parameters among the respective groups.
(2) Serum Lipid Content

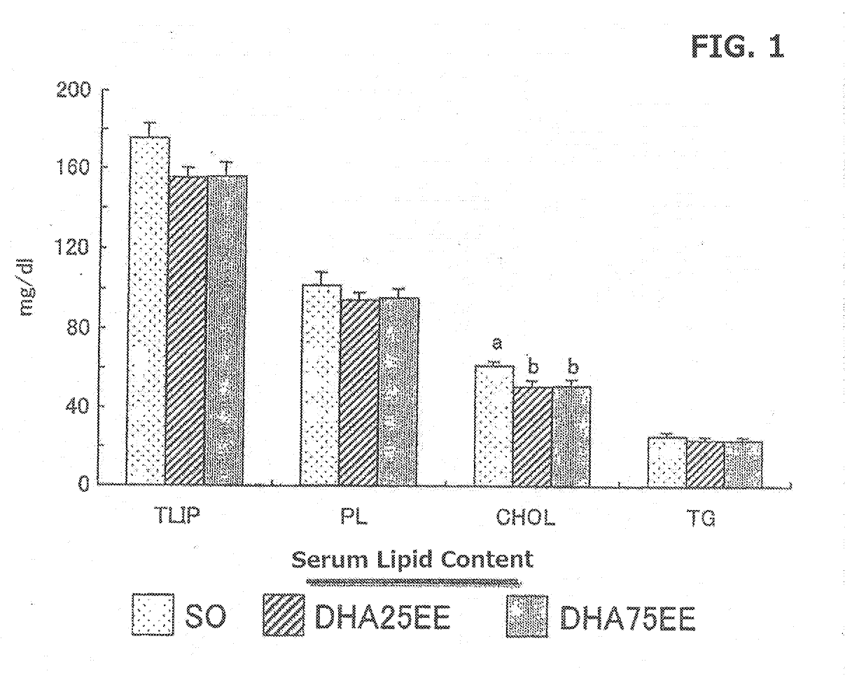

Results are shown in FIG. 1.

In FIG. 1, TLIP denotes total lipid, PL denotes phospholipid, CHOL denotes cholesterol, and TG denotes neutral Lipid. As apparent from FIG. 1, significant differences were observed among different alphabets (feed compositions).
(3) Serum HDL-C and LDL-C Contents

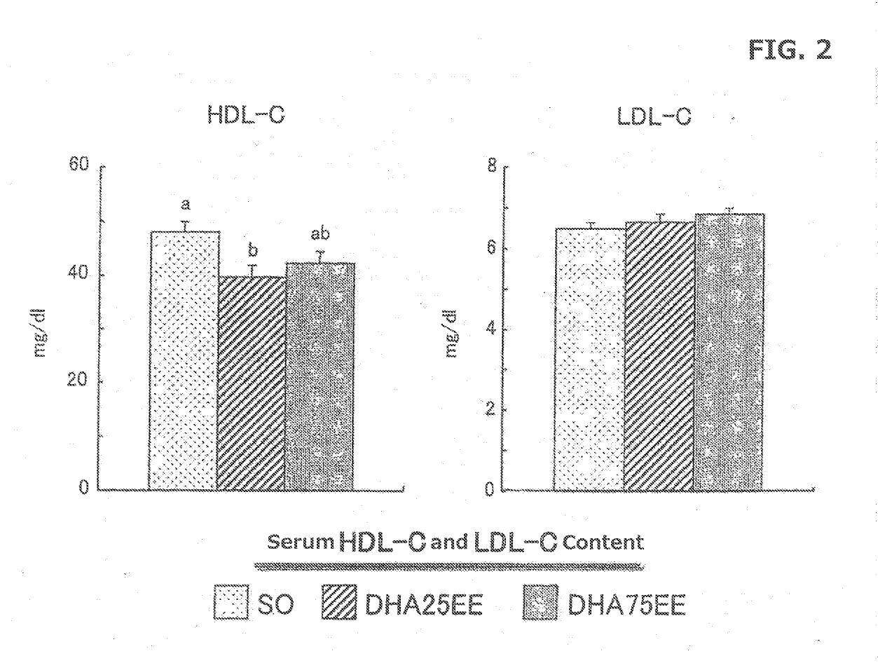

Results are shown in FIG. 2.

In FIG. 2, HDL-C denotes high-density lipoprotein cholesterol, and HDL-C denotes low-density lipoprotein cholesterol.

As apparent from FIG. 2, significant differences were observed among different alphabets (feed compositions).
(4) Hepatic Lipid Contents

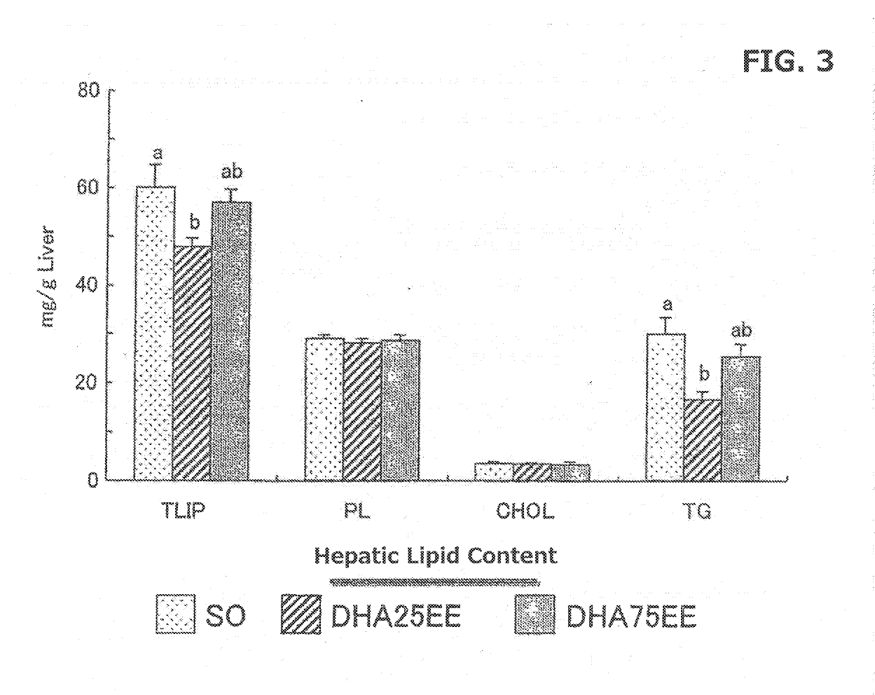

Results are shown in FIG. 3. In FIG. 3, TLIP denotes total lipid, PL denotes phospholipid, CHOL denotes cholesterol, and TG denotes neutral Lipid. As apparent from FIG. 3, significant differences were observed among different alphabets (feed compositions).

### D. Method For Measuring Respective Fatty Acid Amounts of the Liver And Brain, And the Amount of DHA-EE Excreted With Feces

According to the following method, the amount of respective fatty acids in the livers and brains as well as the amount of DHA-EE excreted with feces were measured.
(1) Liver and Brain

The livers and brains are homogenized. Next, total lipid is extracted according to the Bligh & Dyer method. Next, as the internal standard, heptadecanoic acid methyl ester ‘17:0) is added. Next, methyl esterification (60° C., 10 minutes) is performed using 0.5M sodium methoxide. Thereafter, a methyl ester layer is extracted using a silica column, and the measurement is conducted using GCMS (QP2010, Shimadzu Corporation).

Conditions for the GCMS are as follows.
GC
Column: Omegawax-250; Column Temperature: 120° C. to 240° C. (2° C./min); Injector: 250° C.
MS
Ion Source Temperature: 200° C.; Interface Temperature: 250° C.; Detection: Total on Chromatography
(2) Feces

For each group, each day's total feces are powderized using a mill (for seven days). Next, total lipid is extracted in accordance with the Bligh & Dyer method. Next, as the internal standard, heptadecanoic acid methyl ester ‘17:0) is added. Next, an ethyl ester layer is extracted using a silica column, and the measurement is conducted using GCMS (QP2010, Shimadzu Corporation).

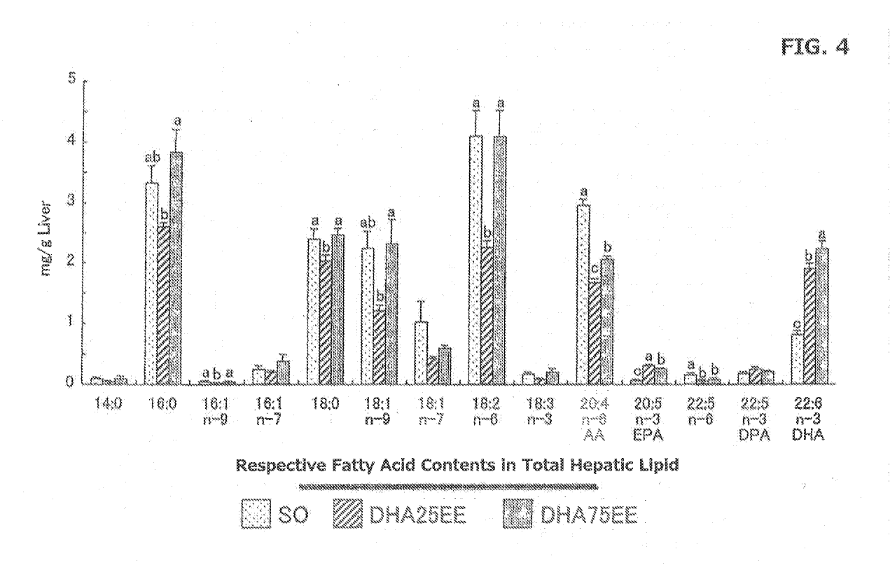

Conditions for GCMS are the same as described above.
(3) FIG. 4 shows Respective Fatty Acid Contents in Total Hepatic Lipid.

In FIG. 4, AA denotes arachidonic acid, EPA denotes eicosapentaenoic acid, DPA denotes docosapentaenoic acid, and DHA denotes docosahexaenoic acid.

The DHA content in the total hepatic lipid was as follows: 2.24 mg/gLiver for DHA75EE; 1.91 mg/gLiver for DHA25EE; and 0.81 mg/gLiver for soybean oil.

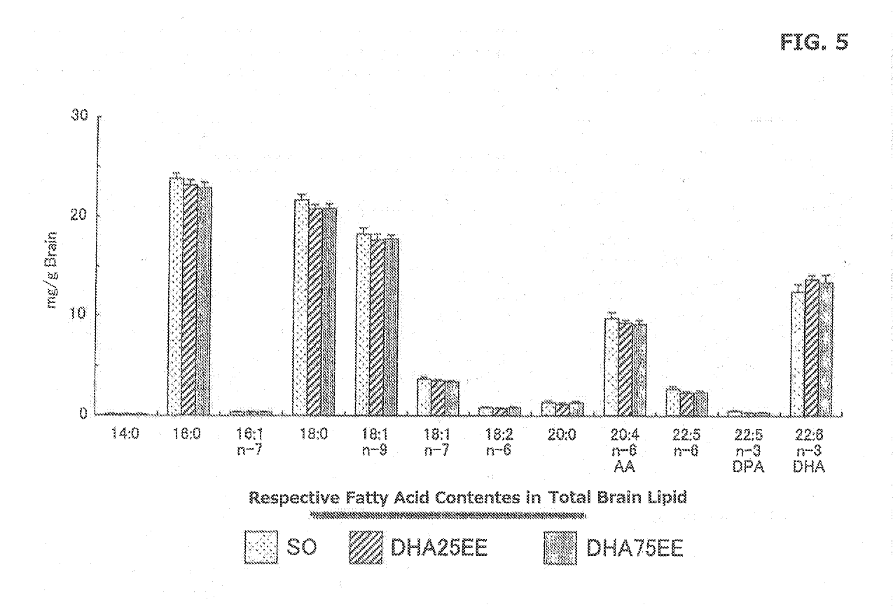

As apparent from FIG. 4, significant differences were observed among different feed compositions.
(4) FIG. 5 shows Respective Fatty Acid Contents in Total Brain Lipid.

In FIG. 5, AA denotes arachidonic acid, EPA Denotes eicosapentaenoic acid, DPA denotes docosapentaenoic acid, and DHA denotes docosahexaenoic acid.

As apparent from FIG. 5, significant differences were not observed among different feed compositions.
(5) Amount of DHA-EE (docosahexaenoic acid ethyl ester) Excreted with Feces

The amount of DHA ester, which was excreted without being absorbed and without being influenced by actions of enzyme, was measured.

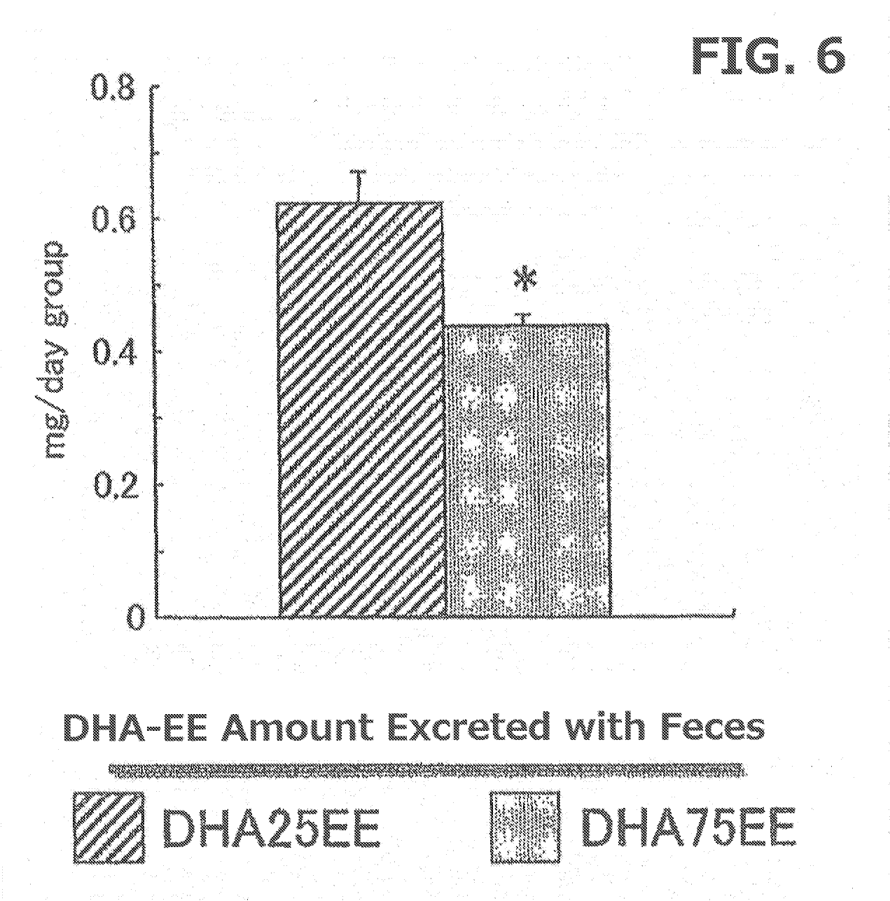

For DHA-25EE, 0.630 mg/day group was excreted. For DHA-75EE, 0.441 mg/day group was excreted. While the same amount of DHA was ingested, more DHA was excreted from the low concentration product. FIG. 6 shows the results.

As apparent from FIG. 6, significant differences were observed between DHA25EE and DHA75EE.

From the foregoing, the following matters are Understood.
(1) in the DHA-EE feed group, the effect of improving serum lipid was confirmed.
(2) In DHA25EE group, reduction in the hepatic TG value was observed. It is assumed that lipid absorbed was low since the amount of the EE-type fatty acid was large in the feed.
(3) In the DHA75EE group, increase in the amount of the hepatic DHA was observed. It is assumed that the absorption rate of DHA is higher for the DHA75EE group since the amount of DHA-EE excreted with feces was higher in the DHA25EE group.
(4) In each group, no significant change was observed in respective fatty acid amounts in the brain. That is, no significant difference was observed in the intake of fluid or feed, or feed efficiency among the experiment groups, and no change was observed in the liver and WAT weight at the dissection.

With regard to the serum lipid composition, there was a tendency of reduced total lipid amount in the DHA-EE intake group compared to the control group, and significant reduction in the total cholesterol was observed in the DHA-EE intake group.

No difference was observed in the serum lipid composition between the DHA-25EE group and DHA-75EE group.

With regard to the hepatic lipid composition, reduction in the total lipid and TG amount was observed in the DHA-EE group compared to the control group, and in particular, strong reduction in the TG amount was confirmed in the DHA-25EE group.

With regard to the DHA amount in the total lipid, significant increase was observed in the DHA-75EE group compared to the DHA-25EE group.

With regard to the total brain lipid DHA, there was a tendency of increase in the DHA-EE feed group compared to the control group, but there was no difference between the DHA-25EE group and the DHA-75EE group.

With regard to the DHA-EE amount excreted with feces, significant increase was observed in the DHA-25EE group compared to the DHA-75EE group. It is assumed that since the amount of fatty acid EE in the feed for the DHA-25EE group was greater than that of the DHA-75EE group, DHA-EE was not influenced by the action of lipase and was thus excreted with feces.

From the present invention, in consideration of the absorption efficiency on the premise of the expression of functionality, it is considered to be more desirable to intake a small amount of high purity DHA-EE than to intake a large amount of low purity DHA-EE.

As described above, the present invention is exemplified by the use of its preferred Embodiments. However, it is understood that the scope of the present invention should be interpreted solely based on the claims. Furthermore, it is understood that any patent, any patent application and any references cited in the present specification should be incorporated by reference in the present specification in the same manner as the contents are specifically described therein.

### INDUSTRIAL APPLICABILITY

According to the present invention, an orally administered composition and a dietary composition can be provided which are used as a pharmaceutical or supplement that comprises a small amount of high purity docosahexaenoic acid ethyl ester.

1. A dietary composition having a serum lipid improving effect, the dietary composition comprising 2.0% by weight or less of a DHA ethyl ester composition comprising 75% by weight or more of DHA ethyl ester.

2. The dietary composition having a serum lipid improving effect according to claim 1, wherein the dietary composition comprises 1.2% by weight or less of the DHA ethyl ester composition.

3. The dietary composition having a serum lipid improving effect according to claim 1, wherein the dietary composition comprises 1.0% by weight or less of the DHA ethyl ester composition.

4. The dietary composition having a serum lipid improving effect according to claim 1, wherein the dietary composition does not substantially comprise a DHA ethyl ester composition comprising 30% by weight or less of the DHA ethyl ester.

5. An orally administered composition having a serum lipid improving effect, the orally administered composition comprising 2.0% by weight or less of a DHA ethyl ester composition comprising 75% by weight or more of DHA ethyl ester.

6. The orally administered composition having a serum lipid improving effect according to claim 5, wherein the orally administered composition comprises 1.2% by weight or less of the DHA ethyl ester composition.

7. The orally administered composition having a serum lipid improving effect according to claim 5, wherein the orally administered composition comprises 1.0% by weight or less of the DHA ethyl ester composition.

8. The orally administered composition having a serum lipid improving effect according to claim 5, wherein the orally administered composition does not substantially comprise a DHA ethyl ester composition comprising 30% by weight or less of the DHA ethyl ester.

9. The orally administered composition according to claim 5, which is a pharmaceutical composition.
